(19) United States
(12) Patent Application Publication
(10) Pub. No.: US 2005/0228441 A1
(43) Pub. Date: Oct. 13, 2005

(54) Biocompatible gripping device

(75) Inventors: John Vivian Wood, Bolnhurst (GB); David Malcolm Grant, Beeston (GB); Sarah Margaret Green, Reagill (GB); David Garlick, Beeston (GB); Timothy Benham, Gothenburg (SE)

Correspondence Address: SMITH-HILL AND BEDELL, P.C., 16100 NW CORNELL ROAD, SUITE 220, BEAVERTON, OR, 97006, US

(73) Assignee: The University of Dundee

(21) Appl. No.: 11/148,781

(22) Filed: Jun. 8, 2005

Related U.S. Application Data
(62) Division of application No. 09/514,759, filed on Feb. 28, 2000.
(63) Continuation-in-part of application No. 08/945,497, filed on Jan. 6, 1998, now abandoned.

(30) Foreign Application Priority Data
Apr. 25, 1995 (GB) 9508328.3

(51) Int. Cl. A61B 17/28

(52) U.S. Cl. 606/205

(57) ABSTRACT

A biocompatible gripping device for surgical use comprises gripping means having at least one deformable gripping element. The element comprises a shape memory material which may comprise functional porosity. The shape memory material may be a shape memory alloy.

### A BIOCOMPATIBLE GRIPPING DEVICE

The present invention relates to biocompatible gripping devices for surgical use. More particularly, but not exclusively it relates to biocompatible surgical needle holders having deformable gripping surfaces.

Surgical needle holders, generally in the nature of forceps, are known and used in both general and laparoscopic surgery. Conventionally, the gripping surface or surfaces of such holders are machined to a knurled finish or coated with a relatively hard and rough coating, for example tungsten carbide, to stop the needle from moving or from dropping out of the needle holder during an operation. Gripping surface of this nature tend to distort the needle and may remove a surface coating from the needle as well as removing part of the needle material itself, which is commonly a type of stainless steel. This distortion and degradation of the needle causes problems, particularly in accurate surgical work. Moreover, it is known occasionally for the needle to be pulled out of the holder during surgery, or to twist within the holder resulting in incorrect alignment of a curved needle.

The present invention seeks to mitigate or obviate these or other disadvantages of the prior art.

According to the invention there is provided a biocompatible gripping device for surgical use including at least one deformable gripping surface.

According to another aspect of this invention there is provided a biocompatible gripping device for surgical use, the device comprising gripping means having at least one deformable gripping element, the element comprising a shape memory material wherein the shape memory material comprises functional porosity.

Preferably the surface or the element is deformable to conform at least in part to the shape of an object gripped thereby to thereby provide enhanced grip of the object. Preferably the surface of the element is formed of a shape memory material, and in particular a shape memory alloy which may return to a non-deformed condition through a shape memory phase transformation upon heating.

The term shape memory material is used herein to refer to a material which recovers from a deformed shape to a pre-formed, substantially stress-free shape on being subjected to certain predetermined conditions.

Preferably the device comprises a pair of co-operating gripping members, each of which provides a gripping surface whereby an article may be held between the surfaces.

Preferably a coating or an insert of the shape memory alloy is provided on each gripping member to form the respective gripping surface.

The shape memory alloy may be a titanium-nickel alloy, preferably a nominally equiatomic alloy, with a composition of desirably between 48-52% atomic % Nickel Titanium. The alloy preferably comprises functional or residual porosity.

The alloy coating or insert may be applied or attached by brazing, soldering, riveting, sintering or compression fit.

Preferably the device is a surgical needle holder, desirably in the form of forceps.

The invention will be further described for the purposes of illustration only with reference to the following accompanying drawings in which:-

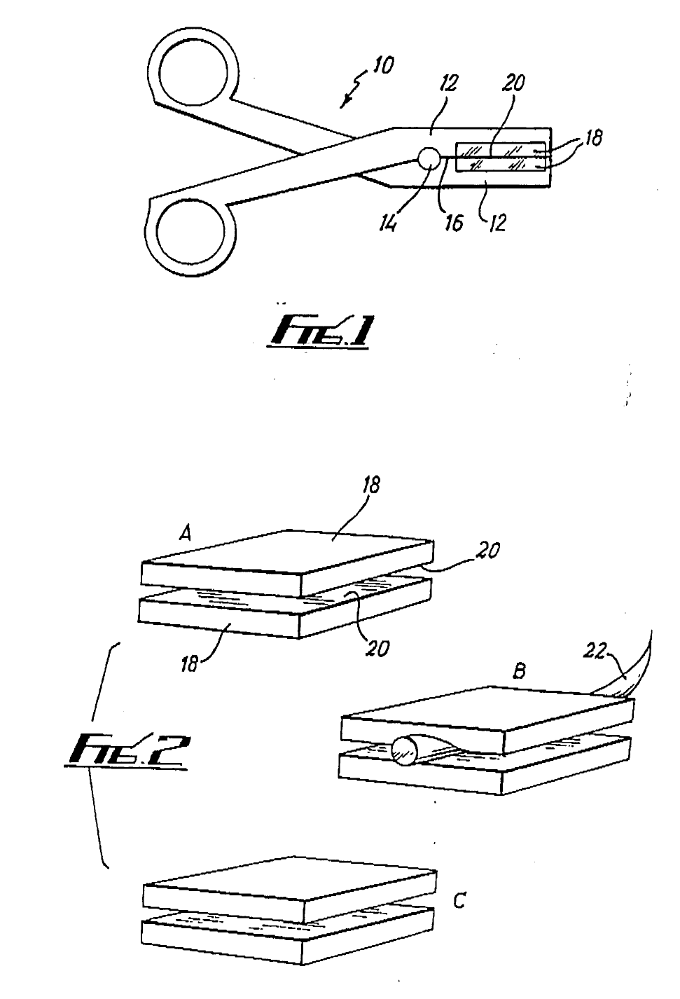

FIG. 1 is a diagrammatic drawing of a surgical needle holder made in accordance with the invention; and

FIG. 2 shows schematically the operation of the jaw inserts.

Referring to FIG. 1, a stainless steel surgical needle holder 10 takes the general form of a pair of forceps. The holder 10 has a pair of jaws 12 movable about a pivot 14. Each jaw 12 has an inner surface 16 in which is provided an insert 18. Each insert 18 provides a gripping surface 20 so that the respective gripping surfaces 20 come into contact with one another when the needle holder is in a closed condition.

Each insert 18 is made from a nominally equiatomic nickel-titanium shape memory alloy, and is formed by a process which will be described hereinafter. The gripping surface 20 provided by the alloy insert 18 is deformable on the application of a force such as may be applied to hold a conventional stainless steel surgical needle 22 in place within the jaws 12 of the needle holder (see FIG. 2B). The inserts 18 thus deform when a needle is gripped enabling a secure and accurate grip to be achieved without damage to the needle itself. The nickel-titanium shape memory alloy has a relatively high coefficient of friction and effectively acts as a sticky material gripping the needle.

The shape memory alloy from which the inserts 18 are formed comprises functional porosity, also known as residual porosity. The functional porosity of the insert allows the device 10 to deform around an article to be gripped such as a needle. This feature in this embodiment has the advantage that it increases the recoverable shape memory deformation from 8% in the prior art cases to about 50%. The functional porosity provides the embodiments described herein with the advantage that the pores allow a greater volume of the insert to be compressed around an object, e.g. a suture needle than would be possible with prior art jaws i.e. jaws that do not comprise functional porosity.

In one embodiment, the shape memory alloy of the inserts 18 are in the martensitic form at room temperature. The compression of the inserts 18 around a needle 22 as shown in FIG. 2 causes the inserts 18 to deform such that they correspond, at least in part, to the shape of the needle 22. The deformation of the inserts 18 around the needle 22 is a plastic deformation and ensures the grip is as accurate as possible, as described above. While not wishing to be restricted to a particular mechanism or theory, it is believed that the deformation of the inserts causes martensitic twinning in the inserts.

The size of the inserts permits several gripping operations to be made before the insert is substantially deformed over its surface. The insert material then requires to be subject to appropriate conditions to cause shape recovery, to return it to its original (undeformed) condition. The nickel-titanium alloy employed in the present example has a martensite to austenite phase transformation temperature occurring between 50° C. and 100° C., and its shape memory effect can therefore be realised either by immersion in hot water or by routine autoclave sterilisation.

Thus, when it is desired to return the inserts 18 to their original non-deformed configuration, the inserts 18 can be heated as discussed above to a temperature of between 50° C. and 100° C. This causes the inserts 18 to return to their original configuration.

In another embodiment, the inserts are in the austenitic phase at room temperature. The deformation of the inserts around the object utilises the superelastic effect, and the inserts recover their original shape on releasing the object. Again it is not wished to be limited to a particular mechanism, but it is believed that the compression of the inserts causes the creation and twinning of stress induced martensite.

It is believed that the pores provided by the functional porosity provide a means of producing an open, extended network of Ni-Ti bridges that can be easily compressed upon squeezing the inserts around an object e.g. a suture needle.

In the present examples, the inserts 18 are produced from elemental pure nickel and titanium powders. The powders are mixed in the approximate ratio 50 at % Ni—Ti, cold compacted and subjected to an inert atmosphere (argon) sinter. The resulting sintered compact contains closed porosity, the extent of which can be controlled by variation of the cold compaction pressure and the initial particle size of the nickel and titanium powders. Modification of the theoretical density of the jaw inserts can thus be achieved. The powder process Ni—Ti intermetallic exhibits the shape memory effect, with a martensite to austenite phase transformation temperature occurring between 50° C. and 100C., dependent upon the composition.

The inserts 18 may be attached to the needle holder either by riveting, soldering, sintering or brazing. The present example employs a type of silver solder, namely a silver-copper-zinc-tin alloy supplied by Eutectic Co. Ltd. of Worcestershire, under the name Superflux 1020. The corresponding flux permits the solder to wet the stainless steel of the needle holder 10 relatively easily. To coat the alloy insert, it was first covered in molten flux, then a small quantity of the solder was melted on it. Oxide forming on the surface was scratched through the solder with an appropriate pointed stainless steel instrument. With the alloy insert held at a suitable degree of super-heat, the solder flowed under the oxide film thus lifting it off. The slag was scraped off and fresh flux applied as protection.

When both surfaces had been coated with solder, they were joined and re-heated until they sweated, ensuring that the correct relative positions were retained.

There is thus provided a surgical needle holder which enables a good grip to be obtained without significant likelihood of damage to the needle.

Modifications may be made within the scope of the invention. In particular, a deformable gripping surface may be provided by other materials than those described, and may be provided on the needle holder in any convenient manner. The invention extends to surgical equipment other than needle holders.

Whilst endeavouring in the foregoing specification to draw attention to those features of the invention believed to be of particular importance it should be understood that the applicant claims protection in respect of any patentable feature or combination of features hereinbefore referred to and/or shown in the drawings whether or not particular emphasis has been placed thereon.

1-14. (canceled)

15. a method of using a biocompatible gripping device that comprises at least one deformable gripping element, the gripping element comprising a material having an austenitic phase and a martensitic phase and having shape memory properties in the martensitic phase, wherein the method comprises gripping an article when the gripping element is at a temperature below the martensite to austenite phase transition temperature and thereby deforming the gripping element from a non-deformed condition to a deformed condition, and thereafter heating the gripping element to a temperature above the martensite to austenite phase transition temperature and thereby returning the gripping element to the non-deformed condition:

16. A method according to claim 15 comprising heating the gripping element to a temperature between 50° C. and 100° C. to return the gripping element to the non-deformed condition.

17. A method according to claim 15 wherein the material comprises a shape memory alloy.

18. A method according to claim 17 wherein the shape memory alloy is a titanium-nickel alloy.

19. A method according to claim 18 wherein the shape memory alloy is a titanium-nickel alloy having substantially 52 atomic % titanium and substantially 48 atomic % nickel.

20. A method according to claim 15 wherein the deformable gripping element is selected from a coating and an insert.

21. A method according to claim 20 wherein the deformable gripping element is applied to the gripping means by brazing, soldering, riveting, sintering or compression fit.

22. A method according to claim 15 wherein the device comprises a pair of co-operating gripping members, each of which includes a gripping surface whereby at least one of said surfaces is provided by said deformable gripping element.

23. A method according to claim 22 wherein each of said gripping surfaces is provided by a respective one of said deformable gripping elements.

24. A method according to claim 23 in the form of a surgical needle holder or forceps.

25. A method of using a biocompatible gripping device that comprises at least one deformable gripping element, the gripping element comprising a material having an austenitic phase and a martensitic phase and having shape memory properties in the martensitic phase, wherein the method comprises gripping an article when the gripping element is at a temperature below the martensite to austenite phase transition temperature and thereby deforming the gripping element from a non-deformed condition to a deformed condition, whereby subsequent heating of the gripping element to a temperature above the martensite to austenite phase transition temperature returns the gripping element to the non-deformed condition.

26. A biocompatible gripping device for surgical use, the device comprising at least one gripping element comprising a material having an austenitic phase and a martensitic phase and having shape memory properties in the martensitic phase, and wherein the gripping element is in the martensitic phase, whereby when the gripping element is used to grip an article, the gripping element is deformed from a non-deformed condition to a deformed condition, and upon heating the deformed gripping element to a temperature above the martensite to austenite phase transition temperature the gripping element returns to the non-deformed condition.

27. A biocompatible gripping device according to claim 26 wherein the martensite to austenite phase transition temperature at which the gripping element can return to the non-deformed condition is a temperature between 50° C. and 100° C.

28. A biocompatible gripping device according to claim 26 wherein the material comprises a shape memory alloy.

29. A biocompatible gripping device according to claim 28 wherein the shape memory alloy is a titanium-nickel alloy.

30. A biocompatible gripping device according to claim 29 wherein the shape memory alloy is a titanium-nickel alloy having substantially 52 atomic % titanium and substantially 48 atomic % nickel.

31. A biocompatible gripping device according to claim 26 wherein the deformable gripping element is selected from a coating and an insert.

32. A biocompatible gripping device according to claim 31 wherein the deformable gripping element is applied to the gripping means by brazing, soldering, riveting, sintering or compression fit.

33. A biocompatible gripping device according to claim 26 wherein the device comprises a pair of co-operating gripping members, each of which includes a gripping surface whereby at least one of said surfaces is provided by said deformable gripping element.

34. A biocompatible gripping device according to claim 33 wherein each of said gripping surfaces is provided by a respective one of said deformable gripping elements.

35. A biocompatible gripping device according to claim 34 in the form of a surgical needle holder or forceps.
